(19) United States
(12) Patent Application Publication
(10) Pub. No.: US 2023/0410947 A1
(43) Pub. Date: Dec. 21, 2023

(54) SYSTEMS AND METHODS FOR RAPID MICROBIAL IDENTIFICATION

(71) Applicant: THERMO FISHER SCIENTIFIC OY, Vantaa (FI)

(72) Inventors: Ping F. Yip, Salem, MA (US); James Stephenson, Raleigh, NC (US)

(21) Appl. No.: 18/247,404

(22) PCT Filed: Oct. 5, 2021
(86) PCT No.: PCT/IB2021/000686

§ 371 Date: Mar. 30, 2023

Related U.S. Application Data
(60) Provisional application No. 63/088,332, filed on Oct. 6, 2020.

(51) Int. Cl. G16B 40/10

(52) U.S. Cl. CPC G16B 40/10

(57) ABSTRACT

Mass Spectrometry has been widely used to identify microbes present in a sample. However, rapid analysis (e.g. 1-5 minutes) of spectral data to identify microbes has proven to be very challenging due to the high level of processing required and complexity associated with identification from a large pool of candidate microbes. Disclosed herein are methods and systems for rapidly identifying microbes present in a sample through the application of conditional likelihoods that certain proteoforms are particularly indicative of a candidate microbe.

### FIELD OF THE INVENTION

The invention relates to mass spectral analysis of samples and methods for rapid classification/identification of microbe species at the genus, species, strain and clone levels.

### BACKGROUND OF THE INVENTION

Mass Spectrometry has been widely used to identify microbes present in a sample. However, rapid analysis (e.g. 1-5 minutes) of spectral data to identify microbes has proven to be very challenging due to the high level of processing required and complexity associated with identification from a large pool of candidate microbes. Typical strategies use what is referred to as a “classifier” approach that utilizes a mathematical model that can predict the likelihood that an unknown sample belongs to a particular class of microbe. The term “classification” as used herein generally refers to the arrangement of organisms into groups (e.g. taxa) on the basis of their similarities and differences.

Classification of microorganisms in clinical microbiology can occur at various levels of granularity. At the genus level, this is considered a group of species with similar phylogenetic and phenotypic characteristics. Species level identification is traditionally thought of as a collection of strains which are more similar to each other than they are to other strains. Classification at the genus species level in molecular clinical biology for any given genus species is defined by ribosomal ribonucleic acid (rRNA) sequence analysis. A finer level of classification can then be obtained at the strain level. The standard definition in clinical microbiology provided by Tenover et al. states that a strain is ‘ . . . an isolate or group of isolates that can be distinguished from other isolates of the same genus and species by phenotypic and/or genotypic characteristics or both’. Finally, at the finest level of classification is what is known as the ‘clone’. In clinical microbiology the clone is defined by Orskov et al. as bacterial cultures that are isolated from different sources, in different locations, at different times, that have many of the same phenotypic and genotypic traits where the identity of said clone is derived from a single origin.

A variety of phenotypic tests have traditionally been used to classify/identify microorganisms in clinical microbiology. Although many of these tests are simple and cost effective, the time to result(s) are lengthy and can have a severe negative impact on patient outcomes. Furthermore, accurate microbial identification and the strain or clone levels typically requires some form of genotypic analysis which may not be cost effective or rapid enough to impact clinical treatment. Genotypic tests also suffer from only determining the “potential” of a given strain or clone to harbor certain resistances or antibiotic susceptibility and do not directly reflect the metabolism of the strain/clone under in vivo or in vitro conditions.

In recent patents and publications, mass spectrometry has proven to be a rapid and accurate method for identifying microorganisms in the clinical environment at the genus species level. Specifically, high resolution/accurate mass analysis of intact protein species directly from individual colonies can in many instances identify microorganisms at the strain level. High mass accuracy allows for the subtle differentiation of protein variants in different strains that may only differ by a single amino acid substitution. This analysis can be performed either directly from peaks found at various m/z ratios produced directly from data acquisition or from the determination of protein molecular weights via a deconvolution algorithm.

Analyzing intact protein mass for microbe identification is important for a number of reasons. One reason includes the fact that the answers generated are useful to guide decisions that are time sensitive. For example, the ability to provide rapid decision making power is particularly important in clinical settings where patient outcomes can be significantly improved.

Most Mass Spectrometry based classification algorithms use aspects of the detected spectrum directly (e.g. use of the detected mass to charge ratio (m/z)) and the intensities of the peaks in the spectrum. Penalty functions are usually constructed based on the difference of the intensities of the peaks of an unknown sample from those in a curated library. Typically, the unknown is identified as the entry in the library with the best match (e.g. match having the smallest penalty).

It is highly desirable to have an analysis approach that substantially increases the speed and performance of processing by a computer in order to provide accurate and rapid microbe identification at the strain and clonal level. For example, increased processing performance completes each task more rapidly thereby freeing up processing resources for other computing tasks that enables rapid and accurate microbe identification at any level of classification. This is particularly important when trying to identify those strains/clones that harbor certain resistance mechanisms or determining the antibiotic susceptibility of said strain/clone against a variety of antibiotics. Identification at the clonal level for example can significantly reduce the number of antibiotic susceptibility tests (AST) needed for rapidly determining patient treatment for a given infection. Since many of the most virulent/resistant clones throughout the world have been extensively characterized, information regarding resistance and antibiotic susceptibility obtained through clonal identification requires just a simple confirmation step to determine patient treatment(s).

### SUMMARY

Systems, methods, and products to address these and other needs are described herein with respect to illustrative, non-limiting, implementations. Various alternatives, modifications and equivalents are possible.

The identification method employed makes use of feature selection combined with standard statistical approaches (i.e. naïve Bayesian, k nearest neighbor, random forest) to identify microbes at the strain and clone level using mass spectra to help improve patient outcomes. The feature selection process is based on the use of F-statistics to identify those features of the mass spectrum that can be emphasized to highlight the differences between closely related strains or for clone determination of a given series of microbes. This additional level of identification can be used to determine microbial resistance as well as guide the antibiotic susceptibility testing process to significantly improve the time to result and improve patient outcomes due to infection.

The above embodiments and implementations are not necessarily inclusive or exclusive of each other and may be combined in any manner that is non-conflicting and otherwise possible, whether they are presented in association with a same, or a different, embodiment or implementation. The description of one embodiment or implementation is not intended to be limiting with respect to other embodiments and/or implementations. Also, any one or more function, step, operation, or technique described elsewhere in this specification may, in alternative implementations, be combined with any one or more function, step, operation, or technique described in the summary. Thus, the above embodiment and implementations are illustrative rather than limiting.

### BRIEF DESCRIPTION OF THE DRAWINGS

The above and further features will be more clearly appreciated from the following detailed description when taken in conjunction with the accompanying drawings. In the drawings, like reference numerals indicate like structures, elements, or method steps and the leftmost digit of a reference numeral indicates the number of the figure in which the references element first appears (for example, element 120 appears first in FIG. 1). All of these conventions, however, are intended to be typical or illustrative, rather than limiting.

FIG. 1 is a simplified graphical representation of one embodiment of a mass spectrometer instrument and a computer that receives information from the mass spectrometer;

FIG. 2 is a functional block diagram of one embodiment of the mass spectrometer and computer of FIG. 1 with an interpretation application in communication with a data structure;

FIG. 3 is a simplified graphical representation illustrating a relationship between protein diversity and the relative abundance;

FIG. 4 is a functional block diagram of one embodiment of a method for determining the identity of an unknown microbe species/strain; and

FIG. 5 is a functional block diagram of one embodiment of a method for selecting a subset of informative proteoform values.

FIG. 6 summarizes the results of the feature selection process for differentiation of 20 strains of E. coli, S. flexeri and S. sonnei.

FIG. 7 is a representative example of the F statistic calculation for the E. coli, S. flexeri, and S. sonnei dataset.

FIG. 8 shows the ability of feature selection to predict resistant S. aureus (MRSA) from 76 different strains using strain identification as a training mechanism.

FIG. 9 demonstrates the ability of feature selection to predict resistant S. aureus from 76 different strains using susceptible/resistant criteria for training.

FIG. 10 compares the results of PBP2a analysis using tandem mass spectrometry (MRSA positive samples) with feature selection to confirm the results generated from feature selection.

FIG. 11 is a representative tandem mass spectrum of the N-terminal sequence of PBP2a from MRSA strains used to confirm the feature selection results.

FIG. 12 shows the differentiation of various susceptible/resistant strains of K. pneumoniae using a twenty minute analysis time with feature selection.

FIG. 13 shows the differentiation of various susceptible/resistant strains of K. pneumoniae using a five minute analysis time with feature selection.

FIG. 14 demonstrates the ability of feature selection to correctly classify susceptible and resistant K. pneumoniae (KPC-2 and NDM-1 positive).

FIG. 15 is a representative KPC-2 tandem mass spectrum used as a direct method to validate the feature selection results for K. pneumoniae.

FIG. 16 demonstrates the ability of feature selection to predict resistant K. pneumoniae from a variety of different strains (susceptible, KPC-2 and NDM-1 positive) using strain based training.

Like reference numerals refer to corresponding parts throughout the several views of the drawings.

### DETAILED DESCRIPTION OF EMBODIMENTS

As will be described in greater detail below, embodiments of the described invention include a substantial improvement in computer processing performance for rapid spectral deconvolution and microbe identification. More specifically, the invention includes using a Naïve Bayes classifier strategy for rapid microbe identification from a large pool of candidate microbes in a complex background. In the embodiments described herein, the microbes may include species and/or strains (e.g. a strain is a variant within a species) of bacteria, yeast, and fungi.

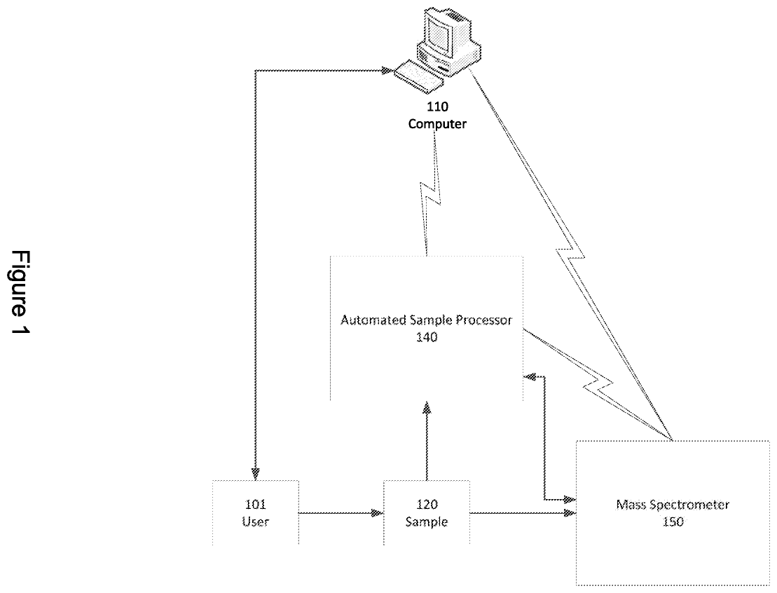

FIG. 1 provides a simplified illustrative example of user 101 capable of interacting with computer 110 and sample 120, as well as network connections between computer 110 and mass spectrometer 150 and between computer 110 and automated sample processor 140. Further, automated sample processor 140 may also be in network communication with mass spectrometer 150. It will be appreciated that the example of FIG. 1 illustrates a direct network connection between elements (e.g. including wired or wireless data transmission represented by lightning bolts), however the exemplary network connection also includes indirect communication via other devices (e.g. switches, routers, controllers, computers, etc.) and therefore should not be considered as limiting.

Also, user 110 may manually prepare sample 120 for analysis by mass spectrometer 150, or sample 120 may be prepared and loaded into mass spectrometer 150 in an automated fashion such as by a robotic platform. For example, automated sample processor 140 receives raw materials and performs processing operations according to one or more protocols. Automated sample processor 140 may then introduce the processed material into mass spectrometer 150 without intervention by user 101. An additional example of an automated platform for processing raw materials for mass spectral analysis is described in U.S. Pat. No. 9,074,236, titled “Apparatus and methods for microbial identification by mass spectrometry”, which is hereby incorporated by reference herein in its entirety for all purposes.

Mass spectrometer 150 may include any type of mass spectrometer that transfers charge to uncharged analytes to produce ions for analysis in order to generate a mass spectrum. Embodiments of mass spectrometer 150 typically include, but are not limited to, elements, that convert analyte molecules to ions and use electric or magnetic fields to accelerate, decelerate, drift, trap, isolate, and/or fragment, to produce a distinctive mass spectrum. Sample 120 may include any type of sample capable of being analyzed by mass spectrometer 150 such as molecules including biological protein samples. It will be appreciated that the term “molecules” include molecules considered to have a “low mass” as well. Some examples of technologies employed by mass spectrometer 150 instruments include, but are not limited to, time-of-flight (e.g. TOF), high resolution ion mobility, ion traping (Fourier transform ion cyclotron resonance (FTICR), Paul traps, or electrostatic trapping devices such as an orbitrap) single/triple quadrupole, or hybrid instruments. An additional example of a mass spectrometer system useable with some or all embodiments of the presently described invention may include the Thermo Scientific Orbitrap™ family of mass spectrometers available from Thermo Fisher Scientific of Waltham, Massachusetts, USA.

Some embodiments of mass spectrometer 150 or automated sample processor 140 may employ one or more devices that include but are not limited to liquid chromatograph, capillary electrophoresis, direct infusion, flow injection all independently or coupled with some form of ion mobility. For example, a chromatograph receives sample 120 comprising an analyte mixture and at least partially separates the analyte mixture into individual chemical components, in accordance with well-known chromatographic principles. The resulting at least partially separated chemical components are transferred to mass spectrometer 150 at different respective times for mass analysis. As each chemical component is received by the mass spectrometer, it is ionized by an ionization source of the mass spectrometer. The ionization source may produce a plurality of ions comprising a plurality of ion species (e.g., a plurality of precursor ion species) comprising differing charges or masses from each chemical component. Thus, a plurality of ion species of differing respective mass-to-charge ratios may be produced for each chemical component, each such component eluting from the chromatograph at its own characteristic time. These various ion species are analyzed—generally by spatial or temporal separation—by a mass analyzer of the mass spectrometer and detected via image current, electron multiplier, or other device known in the state-of-the-art. As a result of this process, the ion species may be appropriately identified (e.g. determination of molecular weight) according to their various mass-to-charge (m/z) ratios. Also in some embodiments, mass spectrometer 150 comprises a reaction/collision cell to fragment or cause other reactions of the precursor ions known as tandem mass spectrometry, thereby generating a plurality of product ions comprising a plurality of product ion species.

Also, in some embodiments mass spectrometer system 150 may be in electronic communication with a controller which includes hardware and/or software logic for performing data analysis and control functions. Such controller may be implemented in any suitable form, such as one or a combination of specialized or general purpose processors, field-programmable gate arrays, and application-specific circuitry. In operation, the controller effects desired functions of the mass spectrometer system (e.g., analytical scans, isolation, and dissociation) by adjusting voltages (for instance, RF, DC and AC voltages) applied to the various electrodes of ion optical assemblies and mass analyzers, and also receives and processes signals from the detector(s). The controller may be additionally configured to store and run data-dependent methods in which output actions are selected and executed in real-time based on the application of input criteria to the acquired mass spectral data. The data-dependent methods, as well as the other control and data analysis functions, will typically be encoded in software or firmware instructions executed by the controller. The term “real-time” as used herein typically refers to reporting, depicting, or reacting to events at substantially the same rate and sometimes at substantially the same time as they unfold, rather than delaying a report or action. For example, a “substantially same” rate and/or time may include some small difference from the rate and/or time at which the events unfold. In the present example, real-time reporting or action could be also described as “close to”, “similar to”, or “comparable to” to the rate and/or time at which the events unfold.

Computer 110 may include any type of computer platform such as a workstation, a personal computer, a tablet, a “smart phone”, a server, compute cluster (local or remote), or any other present or future computer or cluster of computers. Computers typically include known components such as one or more processors, an operating system, system memory, memory storage devices, input-output controllers, input-output devices, and display devices. It will also be appreciated that more than one implementation of computer 110 may be used to carry out various operations in different embodiments, and thus the representation of computer 110 in FIG. 1 should not be considered as limiting.

In some embodiments, computer 110 may employ a computer program product comprising a computer usable medium having control logic (computer software program, including program code) stored therein. The control logic, when executed by a processor, causes the processor to perform functions described herein. In other embodiments, some functions are implemented primarily in hardware using, for example, a hardware state machine. Implementation of the hardware state machine so as to perform the functions described herein will be apparent to those skilled in the relevant arts. Also in the same or other embodiments, computer 110 may employ an internet client that may include specialized software applications enabled to access remote information via a network. A network may include one or more of the many various types of networks well known to those of ordinary skill in the art. For example, a network may include a local or wide area network that employs what is commonly referred to as a TCP/IP protocol suite to communicate. A network may include a network comprising a worldwide system of interconnected computer networks that is commonly referred to as the internet, or could also include various intranet architectures. Those of ordinary skill in the related arts will also appreciate that some users in networked environments may prefer to employ what are generally referred to as “firewalls” (also sometimes referred to as Packet Filters, or Border Protection Devices) to control information traffic to and from hardware and/or software systems. For example, firewalls may comprise hardware or software elements or some combination thereof and are typically designed to enforce security policies put in place by users, such as for instance network administrators, etc.

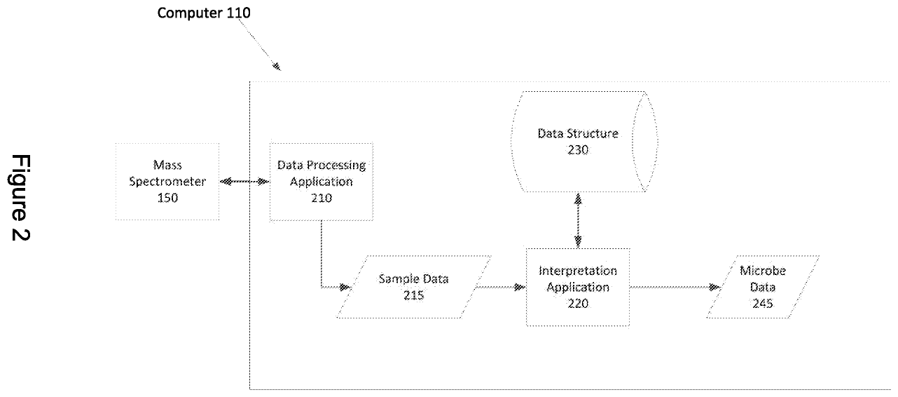

Also, computer 110 may store and execute one or more software programs configured to perform data analysis functions. FIG. 2 provides an illustrative example of an embodiment of computer 110 comprising data processing application 210 that receives raw mass spectral information from mass spectrometer 150 and performs one or more processes on the raw information (e.g. one or more “mass spectra”) to produce sample data 215 useable for further interpretation. For example, one embodiment of data processing application 210 processes the spectral information associated with a material and outputs information such as a known material identified by the analysis of a sample of unknown materials, a value of the mass of the material analyzed (e.g. a monoisotopic mass, or an average mass value), and/or modified spectral profiles from the material (e.g. includes “centroids” that reduces the amount of data needed to characterize the profile). The term “monoisotopic mass” as used herein should be interpreted according to the understanding of those of ordinary skill in the related art and generally refers to the sum of the masses of the atoms in a molecule using the unbound, ground-state, rest mass of the most abundant isotope for each element. Also, the term “centroid” as used herein should be interpreted according to the understanding of those of ordinary skill in the related art and generally refers to a measure used to characterize a spectrum where the centroid indicates where the center of mass is located based on the modeled apex of the profile peak. Additional examples of software program for data processing are described in U.S. Patent Application Publication No. US 2016-0268112 A1, titled “Methods for Data-Dependent Mass Spectrometry of Mixed Biomolecular Analytes”, filed Mar. 11, 2016; and U.S. patent application Ser. No. 15/725,422, titled “System and Method for Real-Time Isotope Identification” filed Oct. 5, 2017, both of which is hereby incorporated by reference herein in its entirety for all purposes.

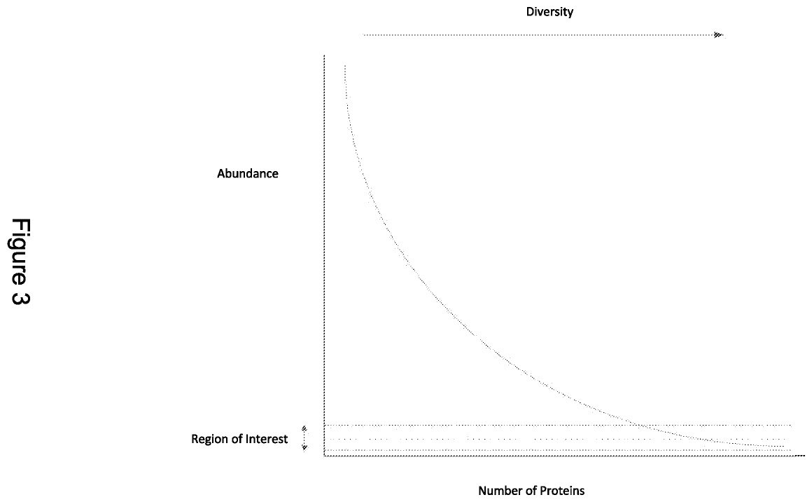

As described above, embodiments of the invention include systems and methods for rapid spectral deconvolution and microbe identification using a classifier approach. Importantly, embodiments of the invention provide substantial improvements in processing capabilities that enable determination of a microbe species/strain from mass spectrometry data in 1-5 minutes. More specifically some embodiments include what may be referred to as a Naïve Bayesian classifier. Those of ordinary skill in the related art appreciate that various Naïve Bayesian classifier strategies have been used in the machine learning fields such as, for example, in the field of text processing (e.g. for spam detection). Also, those of ordinary skill in the related art appreciate that samples may include complex mixtures of different microbe species and/or strains making accurate microbial identification very challenging especially given the high number of possible matches to candidate microbes. For example, samples can have a very high degree of microbe complexity where the signal to noise ratio for a particular protein(s) may be very low. FIG. 3 provides an illustrative example showing that as the number of proteins and diversity increases, the relative abundance of a particular protein decreases making it more difficult to identify.

Quite different from earlier approaches that use the mass spectrum in m/z space directly, embodiments of the presently described invention first perform a deconvolution process on the spectrum to obtain “proteoform” information that may include the molecular weight of each proteoform or protein fragment (e.g. monoisotopic mass of said peak). The term “proteoform” as used herein is often employed in the field of “Top-Down Proteomics” and generally refers to a molecular form of a protein product arising from gene expression. Further, the term “Top-Down Proteomics” as used herein generally refers to identifying and/or quantitating unique proteoforms through the analysis of intact proteins using mass spectrometry and tandem mass spectrometry. The analysis of intact proteins is also sometimes referred to as an “MS1” or single stage mass spectrometry analysis, while “MS2” refers to two stages of mass spectrometry.

In the embodiments described herein the Naïve Bayesian classifier can be applied to MS1 data sets to classify (e.g. identify) unknown species and/or strains of a microbe. This approach works well with spectra of high variance such as mass spectrometry data produced using electrospray ionization techniques (sometimes referred to as “ESI”) from a complex mixture such as a cell lysate. For such data, using intensity values as primary quantities to classify is awkward. For example, it is difficult to quantify intensities below the detection limit, as well as to define a reliable estimate of the variance of intensities for peaks that are close to the detection limit. Furthermore, machine to machine variability tends to introduce more variance to intensities.

Embodiments of the invention also include employing a data structure to store one or more libraries of proteoform information, illustrated as data structure 230 in FIG. 2. Those of ordinary skill in the art appreciate that many types of data structure such as a database could be employed with the presently described embodiments, and thus the description of a library or database data structure should not be considered as limiting. For example, a library of proteoform information may include a likelihood estimate of the relationship of each known microbe species to one or more proteoforms each corresponding to a protein expressed in a microbe species/strain. The likelihood estimate may be experimentally derived and include the frequency of occurrence of each proteoform (e.g. molecular weight M) for proteins identified from a set of replicate samples (e.g. 10 replicates; also sometimes referred to as training sets) of each microbe species/strain (e.g. species B). Or to further refine the granularity of the experiment, frequency could be computed over the scans from a single LC-MS type experiment for each replicate. The term “frequency of occurrence” as used herein generally refers to how often the proteoform value occurs for that microbe species/strain and may be expressed in terms of percentage (e.g. 1%), fraction (e.g. 1/100), decimal (e.g. 0.01) or other notation known to those of ordinary skill. In the present example, likelihood estimates may be mathematically represented asP(M|B) (e.g. in Bayesian terms P(M|B) represents the conditional probability of observing molecular weight M given that it is microbe species B (also stated as species B “is true”)). In the present example, the library of proteoform information may be constructed for proteins associated with known microbes using the processes described herein.

Those of ordinary skill in the related art appreciate that Bayes theorem describes the probability of an event based on prior knowledge of conditions that might be related to the event. In the described embodiments Bayes theorem may be mathematically represented as:

$$P ( M \vert B ) = \frac{P ( B \vert M ) P ( M )}{P ( B )} \qquad Equation 1$$

where:
P (M|B) and P(B|M) are conditional probabilities as described above
P(M) and P(B) are the ‘a priori’ probabilities of observing M and B independently of each other
   In practice, we would like to determine the probability that an unknown sample is of a specific species/strain/clone given the occurrence of a set of proteoforms observed in an experimental assay. Inverting equation 1, we have the conditional probability desired.

P(B|M)=P(M|B)P(B)/P(M) Equation 2

For multiple proteoform assays such as obtained with mass spectrometry, M will actually be a combination of multiple proteoforms M1, M2, . . . Mi, . . . , Mn. The quantity P(M|B) is experimentally determined when we compile the library.

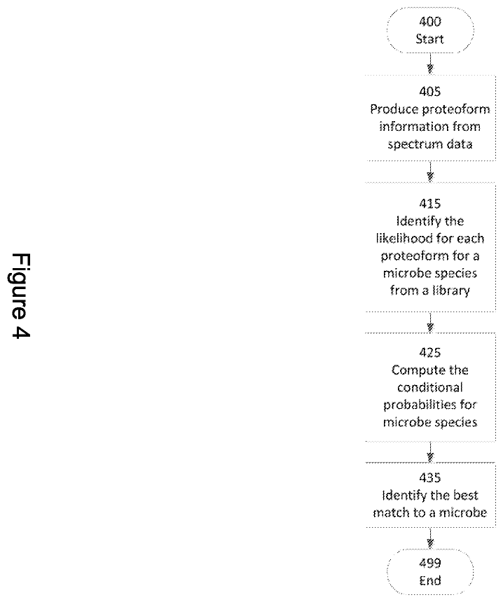

FIG. 4 provides an illustrative example of an overview of one embodiment of the invention that identifies for an unknown microbe species/strain (S) in sample 120. Also some embodiments of the invention produces a score corresponding to the confidence level of the identification. As illustrated in step 405 computer 100 first has data processing application 210 perform a protein deconvolution step to produce sample data 215 comprising the proteoform information from the spectrum data derived from sample 120 by mass spectrometer 150.

Subsequently, in step 415 interpretation application 220 identifies the conditional likelihood of P(Mi|B) from the library in data structure 230 for some or all of the proteoform values (Mi), wherein i stands for the i-th proteoform identified from sample 120. It will be appreciated that the proteoforms values may typically identify multiple candidate microbe species/strains from the library (e.g. microbe species/strain B, C, D, etc.). In some embodiments, the library may include every proteoform value associated with each known microbe species/strain or microbe species/strain of interest. However, in an alternative embodiment the library may only include proteoform values that have been determined as “informative” for identifying the corresponding microbe species/strain. For example, as will be described in further detail below in regard to “feature selection”, in some embodiments only a selected subset of individual likelihoods associated with the most informative proteoform values may be employed to improve the performance and accuracy of the classifier strategy.

Then, as illustrated in step 425 interpretation application 220 computes the conditional probability P(B|M1, M2, . . . Mi, . . . ) for each candidate microbe species/strain identified in step 415, using equation 2 and the empirically established library for P(M1, M2, . . . |B). Furthermore, in almost all applications, one assumes conditional independence of Mi to arrive at P(M1, M2, . . . B) being equal to P(M1|B) P(M2|B) . . . P(Mn|B)−substituting 1−P(Mi|B) for P(Mi|B) for the absence of Mi. Finally, P(M1, M2 . . . Mn) can be computed easily from the library as the product of frequency of occurrences of M1, M2 . . . etc, while P(B) is usually assumed to be the same for all microbes (equal priors)

Finally, in step 435 interpretation application 220 identifies the microbe species and/or strain that has the highest conditional probability computed from equation 2, amongst all microbe entries in the library, as the most likely candidate for the unknown microbe. Interpretation application 220 then outputs the identification as microbe data 245 which may also include other information such as the conditional probability of the best candidate microbe. In some embodiments computer 110 may also provide the identification to user 101 via a display (e.g. a graphical user interface) and/or email, text, or other form of electronic transmission.

It will also be appreciated that although FIG. 2 illustrates data processing application 210 and interpretation application 220 as separate elements, the functions of both application 210 and 220 as described herein may be performed by a single application. Further some functions described as performed by application 210 may be performed by application 220 and vice versa. Therefore the example illustrated in FIG. 2 should not be considered as limiting.

In some embodiments a sample may not produce sufficient proteoform information for effective identification of the microbe species/strain. This can occur in situations when experimental conditions are compromised (spray failure, poor MS calibration, etc.). In the described embodiments it may thus also useful to include a negative control in the library such as, for example, a fictitious microbe species that has zero likelihood of correspondence to any proteoform value in the library. When an unknown microbe species/strain matches the negative control better than any of the other entries in the library, then the unknown microbe species/strain is classified as a no call. Also, in the same or alternative embodiments comparing a 0 likelihood value to another 0 likelihood value is appreciated by those of ordinary skill as an ill-defined mathematical operation that can confound the analysis. Therefore, in some embodiments it may be useful to replace the 0 likelihood values in the library with some small value (e.g. the value may be some arbitrary value that is >0 and <1, such as 0.23) and to replace the 1 values with a 1 minus that small number value.

As described above, some embodiments of the invention may be further enhanced using what may be referred to as “Feature Ranking” and “Feature Selection” approaches. For example, feature selection includes a process whereby a suitable subset of one or more features (e.g. proteoform markers) is selected to optimize the performance of the classifier. For multi-marker problems, it is often the case that some proteoform markers are more informative than others. Weeding out less informative and potentially noisy and confounding proteoform markers can substantially improve the performance of the classifier. As will be described in greater detail below the subset of proteoform markers used with the classifier can be identified using “training data” typically derived using the same experimental conditions employed for the identification of the unknown microbe species. For example, if frozen samples are used for the test for the unknown microbe species, then the training data should similarly be derived from frozen samples.

Also, feature selection of the suitable subset is typically based on a feature ranking of each proteoform marker according to the information content for the proteoform marker. The information content of a proteoform marker can be calculated in a number of ways, such as for example by what is sometimes referred to as a “resampling” approach (specific resampling approaches may include what is referred to as a “randomization test” or a “permutation test”). This process may sometimes also be referred to as determining the “importance” of a proteoform marker. In the presently described example, a value for the proteoform marker may be observed over a plurality of training samples, then the observed values can be randomized and evaluated. A drop in performance due to randomization can then be used as a measure of importance where the greater the degree of drop corresponds to a corresponding greater degree of importance.

The importance values can then be used to rank the proteoform markers. For example, many different combinatorial approaches are known that can be used to assess the list of ranked markers to finalize a selection of the desired subset. One such approach includes use of the top N ranked markers to build models, where N can be determined by a resampling procedure. Alternatively, the performance can be monitored as a function of rank and aggregate by rank, keeping only those markers that provide performance improvement. N, the optimal number of top markers/proteoforms, varies significantly depend on the data set. It can vary from being a tenth of the total number of markers to being close the total number. Typically, the more the proteoforms detected, the smaller (relatively) the N is, as most of the proteoforms tend to be noisy and confounding.

However, there are drawbacks to the resampling feature ranking approach described above. First, using a resampling strategy to estimate feature importance is computationally intensive, demanding significant processing resources from computer 110. In particular, for problems with potentially tens of thousands of proteoform markers, as is the case with high resolution ESI mass spectrometry, this approach is not computationally efficient. Compounding the inefficiency problem is the fact that, a resampling approach is completely dependent on the model/classifier building process; any change in the parameters will necessitate a completely new ranking computation from scratch. Another problem associated with a resampling strategy occurs when many of the proteoform markers are highly correlated. Correlation of proteoform markers is a common occurrence for Mass Spectrometry profiling of complex samples. For example, what is referred to as “Adduction” includes protein modifications such as oxidation and formylation that introduce sets of highly correlated peaks into the data. In addition, a lot of proteins from a complex sample tend to be co-expressed in different microbe species/strains and thus exhibit high a degree of correlation. Using the resampling—randomizing strategy to estimate importance tends to under-estimate the importance of proteoform markers that are correlated with many other proteoform markers. Finally, a combinatorial approach of selecting from a ranked list of markers runs the risk of over-fitting in which the data set is over-used to create a biased classifier.

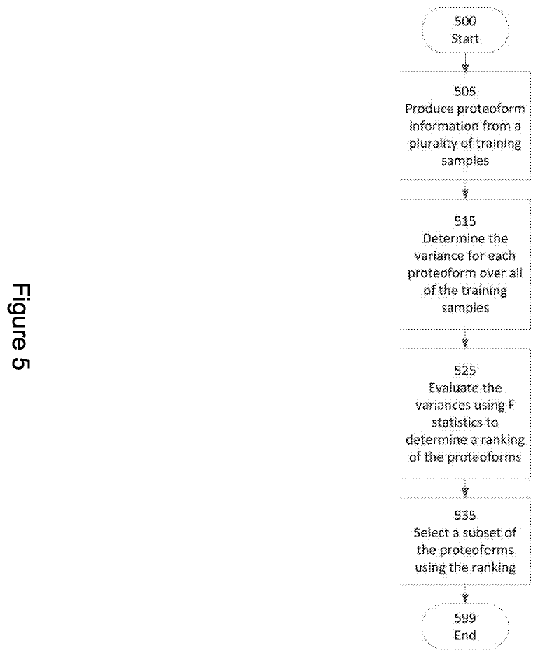

Therefore, embodiments of the presently described invention include improved approaches to feature ranking and feature selection over the resampling based approach described above. Importantly, the feature selection strategy of the presently described embodiments provide the greatest benefit for distinguishing microbe species/strains that are closely related and are difficult to resolve from each other (e.g. have a high degree of similarity of the proteoform markers). FIG. 5 provides an illustrative example of a method of feature ranking and feature selection according to some embodiments of the described invention. As illustrated in step 505 computer 100 first has data processing application 210 perform a protein deconvolution step to produce sample data 215 comprising the proteoform information from a plurality of samples 120 for training by mass spectrometer 150. For example, training samples may each include different microbe species/strains and/or include some number of replicates of microbe species/strains.

In some embodiments the improvement includes use of an independent statistical measure to perform feature ranking of the proteoform markers. As described above, some embodiments of the Naïve Bayesian model utilize the frequency of one or more proteoform markers over a number of samples. Therefore, the variance of occurrence for each proteoform marker can be easily computed over all of the samples. As illustrated in step 515, interpretation application 220 calculates the variances and as illustrated in step 525 computes what is referred to as the “F statistics” for each proteoform marker (also sometimes referred to as an “F-test”) for the samples in the training data. In general F statistics are useful for comparing models that have been fit to a data set to identify the model that is a best fit to a statistical population that the data was sampled from. There are a number of F statistics tests known to those of ordinary skill in the art.

In the embodiments described herein, F statistics of a proteoform marker may include a measure of how well the training samples are differentiated from each other based on that proteoform marker alone. For example, the statistical test referred to as “Analysis of Variance” (e.g. ANOVA) is based on the F statistics and can be employed for feature ranking. In the present example, the ANOVA test can be used as a measure of the importance of markers, where the higher the degree of the F statistic value correlates to a similarly high degree of discriminatory power of the proteoform marker. A ranking of the proteoform markers can be sorted by decreasing F statistics (e.g. in a table or other representation).

In the embodiments described herein, the F statistics are extremely efficient to compute and are completely independent of the modeling approach. Furthermore, since the F statistics are computed for each proteoform marker independent of others, the complication due to marker correlation is avoided. It will also be appreciated that other statistical measures could also be used to rank markers, such as entropy or RSD of feature frequencies, which yield similar performance.

Then, as illustrated in step 535, the F statistics ranking described above can be utilized for feature selection. In some embodiments, the F statistics table of proteoform markers sorted by decreasing F statistics can be used without incurring significant computational overhead to evaluate the performance of the Naïve Bayesian model as a function of the number of cumulative markers used. To determine the F statistics cutoff to use for feature selection, for example, one performs a standard model building exercise but with a test set to gauge the performance of the model/classifier. The accuracy of the model against the test set can be tracked as a function of F, for successively more features (ranked by the F statistics), aggregated. The cutoff value for the F statistics is then chosen as the value at which the test accuracy attains an optimum. In addition, other metrics other than the overall accuracy can be used, such as specificity, accuracy for a particular microbe to select the cutoff. Finally, to improve the reliability of the determination of the cutoff, one can use a resampling strategy to obtain an average optimal cutoff. It should be pointed out that this resampling strategy is not used to calculate the importance of the markers as in other approaches, the importance has already been determined by the F statistics. It is used merely to obtain a more robust estimate of the cutoff. For example, as described above the correlation of different markers according to certain criteria such as the various oxidation states of a single protein can be problematic. However, it is advantageous to use only the most diagnostic peak from the correlated group as measured by the F statistics, and ignore the others.

In one embodiment, interpretation application 220 may use correlation information during the feature selection process by implementing a filtering method. For example, during feature selection when interpretation application 220 selects the aggreate markers beginning with the highest ranked marker, for each new proteoform marker interpretation application 220 screens the correlation coefficient against all the proteoform markers previously selected to determine that it is equal to or below a certain threshold value. If the threshold correlation coefficient value of any proteoform marker is above the threshold value then that proteoform marker fails the correlation test and interpretation application 220 excludes the proteoform marker from consideration. In the present example, interpretation application 220 evaluates each proteoform marker in the F statistics table of proteoform markers. Further, interpretation application 220 determines performance as a function of the number of aggregated proteoform markers which pass the correlation test. The threshold value, in one embodiment, could be considered a tunable parameter, which can be optimized for better model performance.

In the same or alternative embodiments interpretation application 220 may not only provide a single prediction score for each test but also the prediction scores of the close runners up as well. As described above, interpretation application 220 calculates the conditional probability using the Naïve Bayesian model P(B|M1, M2, . . . Mi, . . . ) for each candidate microbe (B) in the database given the appearance of markers M1, M2 . . . in a test measurement, where the B that maximizes the conditional probability is chosen as the winning prediction. Interpretation application 220 can simply report back the conditional probability P as a score, however in some embodiments it may be desirable to use log(P) as a score. Also, user 101 can specify the number of runners up desired for each test classification and computer 110 will provide a list of the runners up and their associated scores (e.g. in a Graphical User Interface).

For example, a numerical score may be highly desirable in situations where a more quantitative prediction is required. One such situation may include what is referred to as “hetero-resistance” that occurs when a subpopulation of a microbe species/strain is not susceptible to an antibiotic while the majority of the population is. In the case of hetero-resistance the failure of detecting a targeted marker is not sufficient to indicate susceptibility but using the detection of other indirect markers could indicate resistance. Having a numerical score can help fine tune the score cut off to allow reliable prediction of resistance indirectly. Another situation may include what is referred to as “multiple resistance” that occurs when one or more microbe species/strains are resistant to multiple antibiotics. For such cases, a numerical score associated with each resistance prediction could help indicate multiple resistance instead of just the most likely resistance mechanism.

### EXAMPLES

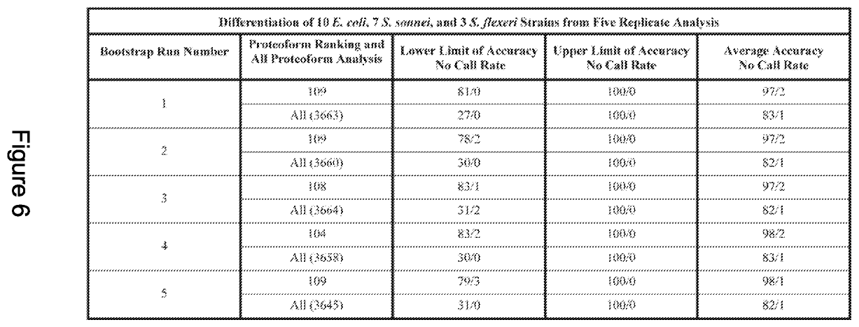

In FIG. 6 is an example of applying the feature selection method, without the correlations filter, to a strain differentiation problem. Briefly, the 30 minute single stage mass spectrometry (MS1) liquid chromatography mass spectrometry (LC-MS) data of 10 E. coli, 7 S. sonnei and 3 S. flexeri strains were collected in 5 fold replicates. The raw mass spectra were deconvoluted to obtain proteoform monoisotopic masses. The proteoform mass values form the feature set for the Naïve Bayesian classifier. A 100 fold bootstrap resampling was performed using 4 replicate for training and 1 for testing. The bootstrap was repeated 5 times to arrive at the data shown in FIG. 6.

The first column contains the run number of the five independent bootstrap runs. The cumulative rank (F statistic) of the markers used for the prediction results are listed in the proteoform ranking column. Two performance numbers were presented: one at the optimal cumulative rank, and two, for all markers available (the number in parenthesis is the total number of ranks for the marker set). The performance for the best and worst strain identification is listed in lower and upper limit columns respectively as percentages. In the current example, 78/2 translates to 78% accuracy and 2 percent no call. Finally the performance averaged over all the 20 strains are listed in the “Average” column.

The performance at the optimal cumulative rank is consistently at 97 or more percent accurate with 2 percent no call, whereas the performance for all marker, i.e. without feature selection, is consistently at 82 percent with 1 percent no call. The feature selection step translates to a 15 percent performance gain.

Based on studies on other data sets, the performance gain using feature selection ranges from minimal (under 5 percent) to very significant (over 20 percent). In general, as one would expect, the more the number of features the more feature selection will improve the classification result.

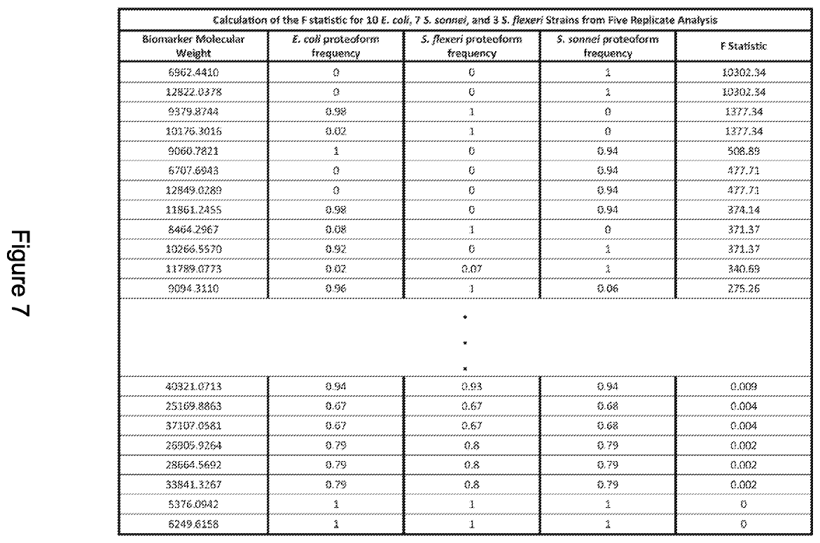

In FIG. 7 is shown the representative F statistic calculations for the E. coli, S. flxeri, and S. sonnei dataset described in FIG. 6. The data are arranged by significance (highest F statistic calculation) based on the frequency data shown in FIG. 7. The corresponding molecular weight of the protein markers is in the left most column. The first 12 entries in the figure are those markers with the highest significance, and the last 8 entries are for those markers with the least discriminating power in the dataset. In general, the observed distribution curve for the F statistic yields a sigmoidal shape with the slope of curve dependent on the relatedness of the species considered.

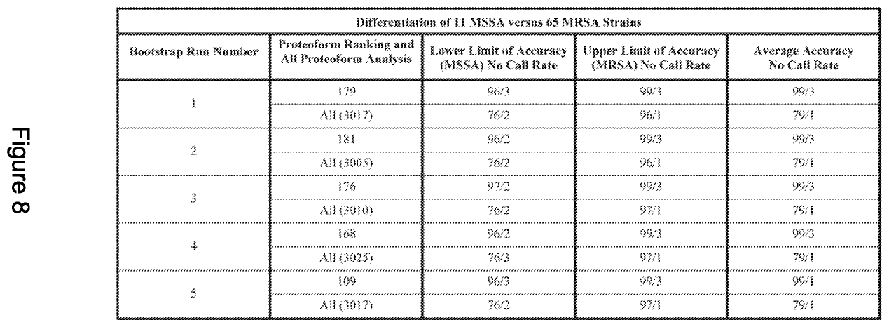

The clonal identification process is also very effective in working with large datasets which can be trained in a variety of ways to answer specific microbial identification questions or clinical outcomes. In FIG. 8 is shown the clonal identification results for 11 susceptible and 65 resistant for of S. aureus. In total 435 samples were analyzed with 6 replicates per strain from actual patient samples. This included 54 protein standards to check instrument performance, 28 blank samples, and 15 quality control runs to ensure data integrity. The proteoform mass values form the feature set for the Naïve Bayesian classifier. A 100 fold bootstrap resampling was performed using 4 replicates for training and 1 for testing. The bootstrap was repeated 5 times to arrive at the data shown in FIG. 8. The training set in FIG. 8 was based on strain identification and the ability of this model to predict resistant/susceptible S. aureus for patient treatments associated with potential MRSA infections.

The novel aspect of this approach is that the protein PBP2a (associated directly with MRSA) was not used in any way to predict and identify the S. aureus strains as susceptible or resistant. As demonstrated in FIG. 8, the use of feature selection (using the F statistic) resulted in an overall improvement in classification accuracy of 20 percent. Using feature selection the average accuracy for identifying an S. aureus strain as MRSA was 99 percent. By not employing feature selection, results were significantly worse with an overall success rate of 79 percent.

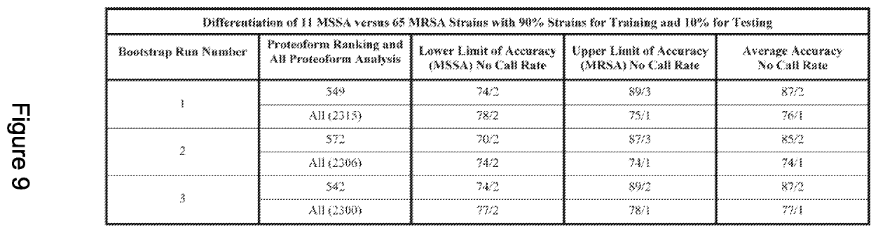

Another model was constructed from the aforementioned S. aureus dataset by training on 90 percent of the data for PBP2a negative/positive to represent susceptible/resistant strains in predicting patient treatment options. The remaining 10 percent of the data was used for the test case. Three separate bootstrap runs were employed to ensure no bias in the results. The data summarized in FIG. 9 yields a 12 percent improvement using feature selection over equal weight applied to the protein markers observed. The average success rate with this model was 87 percent compared to only 75 percent for unweighted data as shown in FIG. 9.

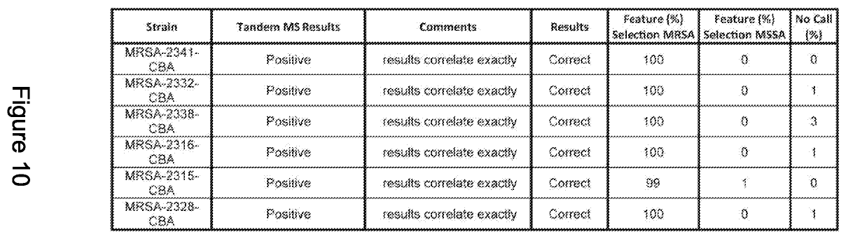

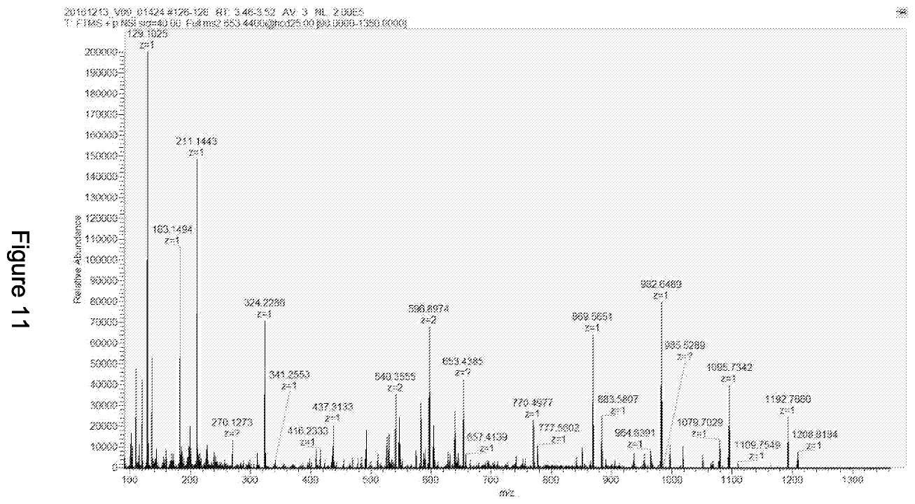

To prove the models work for the approach described above for the determination of susceptible versus resistant strains of S. aureus using feature selection, random strains were picked for comparison to direct detection using tandem mass spectrometry results for the presence of the PBP2a protein. Six different strains were run with feature selection for MRSA positive/negative (methicillin susceptible S. aureus—MSSA) analysis as shown in FIG. 10. In each case, the feature selection results were verified with the tandem mass spectrometry data which confirm the N-terminal sequence of PBP2a (see FIG. 11).

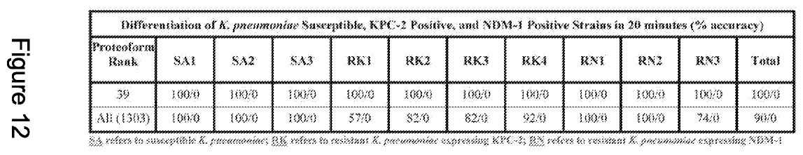

In order to check the performance of the feature selection for strain identification for rapid analysis runs and using different numbers of protein markers, a dataset comprising known Gram negative bacteria (many of which are carbapenemase resistant enterobacteriaceae—CRE) was analyzed. This dataset comprised three susceptible, four KPC-2 positive, and three NDM-1 positive strains of K. pneumoniae. The first analysis conditions consisted of 20 minute analysis runs of 5 replicates each of the various K. pneumoniae strains. The results shown in FIG. 12 demonstrates 100% accuracy for strain identification using feature selection for all susceptible and resistant bacteria. This result was obtained using only 39 protein markers derived from the F statistical calculations of feature selection. In comparison, unweighted results demonstrated excellent accuracy for the classification of susceptible strains (100 percent), but only 57 to 82 percent accuracy for KPC-2 positive and 74 to 100 percent accuracy for NDM-1 positive strains.

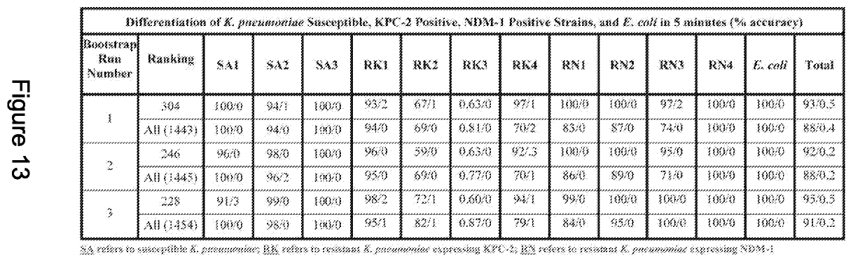

To improve patient treatment options with CRE, rapid analysis times are critical for increasing survival rates not just for pathogen identification, but for the presence of specific CRE markers. Using the aforementioned K. pneumoniae dataset, analysis times were decreased to 5 minutes and featured selection again was compared directly to unweighted analysis for strain identification. The results in FIG. 13 produced improved performance for feature selection across the three bootstrap analysis of the five minute data all with average accuracies of over 90 percent for each bootstrap run (see last column on the right in FIG. 13).

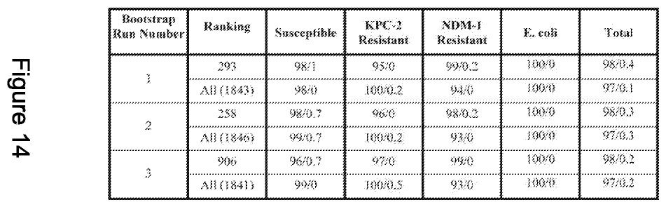

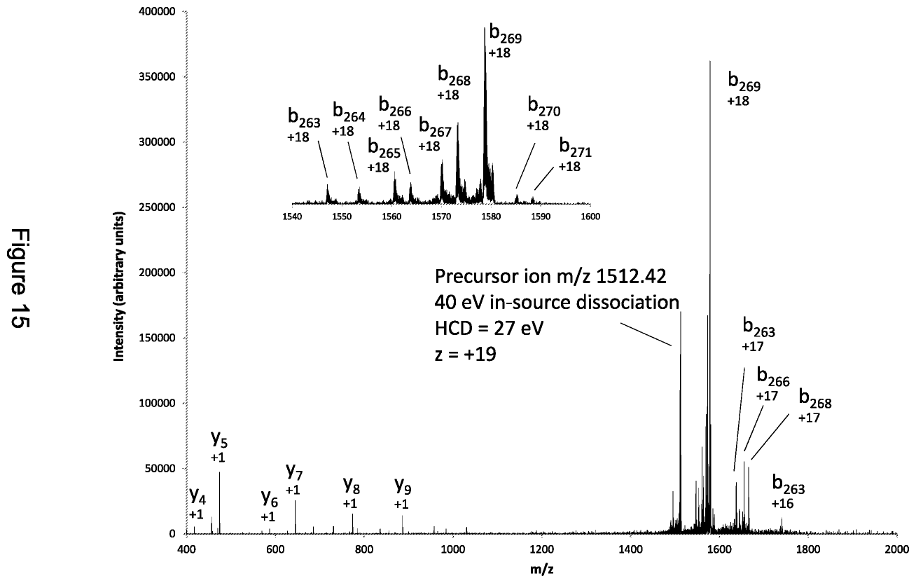

In order to expand the capabilities of resistance detection beyond the MRSA example illustrated in FIG. 11, the aforementioned K. pneumoniae dataset was trained for detection of susceptible KPC-2 positive and NDM-1 positive strains. The individual strain classification results shown in FIG. 14 have accuracies that range from 95 to 100 percent. In order to provide evidence of the robustness of the approach, an additional E coli samples was analyzed in order to try and introduce confounding factors into the method. As shown in FIG. 14, all E. coli samples were distinguished from the susceptible and resistant forms of K. pneumoniae. As with the MRSA example, results from feature selection were compared directly with tandem mass spectrometry results searching for the individual resistance markers. In all cases for the KPC-2 examples the resistant protein was detected successfully (see corresponding verified tandem mass spectrometry data in FIG. 15).

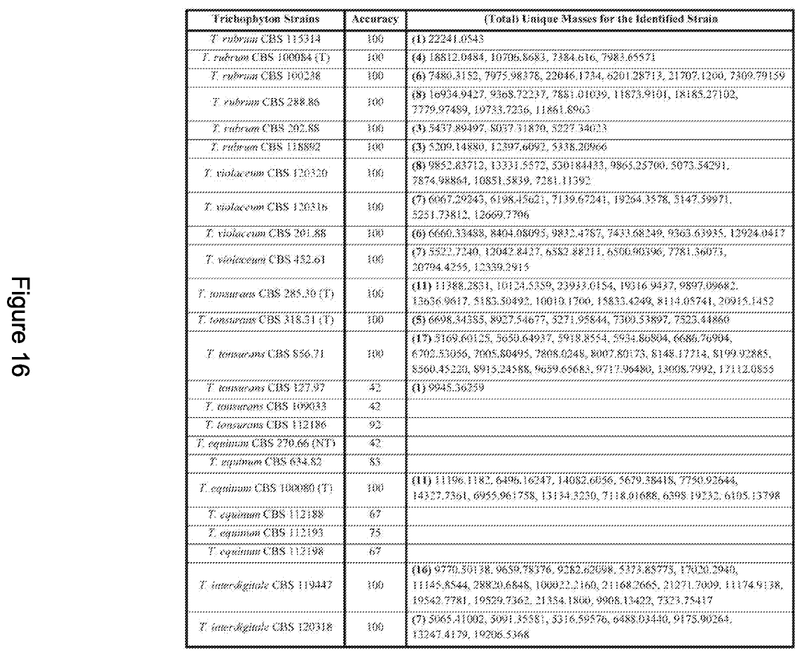

To check the validity of the approach using data from more complex organisms, a series of Trichophyton strains (pathogenic eukaryotic fungi) was analyzed using the feature selection approach. Here we analyzed 24 strains of closely related dermatophytes were subjected to the feature selection approach. Three species were identified correctly down to the strain level (T. rubrum, T. violaceum, and T. interdigitale), while in the T. tonsurans-equimum complex eight of the 12 strains showed nearly identical proteomes, indicating an unresolved taxonomic conflict apparent from previous phylogenetic data. In FIG. 16 is shown the results of the proteomic data with feature selection. The number of unique proteins and protein masses corresponding to each strain are listed in the column on the far right of FIG. 16 along with the individual accuracies of the strain classification approach.

Having described various embodiments and implementations, it should be apparent to those skilled in the relevant art that the foregoing is illustrative only and not limiting, having been presented by way of example only. Many other schemes for distributing functions among the various functional elements of the illustrated embodiments are possible. The functions of any element may be carried out in various ways in alternative embodiments.

What is claimed is:

1. A method for identifying a microbe species, comprising:
determining a plurality of proteoform values from spectral information derived from mass spectral analysis of a sample comprising an unknown microbe species;
for one or more of the proteoform values identifying a likelihood the proteoform corresponds to a particular microbe species, wherein the proteoform value belongs to a subset of informative proteoform values for the candidate microbe species;
determining a conditional likelihood for a plurality of candidate microbe species using the identified likelihoods for each proteoform;
identifying the conditional likelihood of the candidate microbe species that is a best match to the unknown microbe species.

2. The method of claim 1, wherein,
the subset of informative proteoform values is determined using the proteoform values from a plurality of training samples.

3. The method of claim 2, wherein,
the proteoform values from the plurality of training samples are derived under the same experimental conditions as the plurality of proteoform values from the unknown microbe species.

4. The method of claim 2, wherein,
the training samples comprise samples from different candidate microbe species.

5. The method of claim 2, wherein,
the training samples comprise a replicate sample from at least one of the candidate microbe species.

6. The method of claim 2, wherein,
the subset of informative proteoform values are selected using the method comprising:
determining a variance value for each proteoform over all of the training samples;
ranking the variances of the proteoform values using an F statistical test; and
selecting the subset of informative proteoform values from the ranking.

7. The method of claim 6, wherein,
the F statistical test comprises an analysis of variance test.

8. The method of claim 1, wherein,
the sample comprises a complex mixture.

9. The method of claim 8, wherein,
the complex mixture comprises a cell lysate

10. The method of claim 1, wherein,
the proteoform value comprises a mass value.

11. The method of claim 10, wherein,
the mass value comprises a monoisotopic mass value.

12. The method of claim 1, wherein,
the unknown microbe species are selected from the group consisting of bacteria, yeast, and fungi.

13. The method of claim 1, further comprising,
providing an identification of the candidate microbe species that is the best match to a user.

14. The method of claim 13, wherein,
the identification comprises a score.

15. A system for carrying out the method of claim 1.
